(19) United States
(12) Patent Application Publication
(10) Pub. No.: US 2013/0156902 A1
(43) Pub. Date: Jun. 20, 2013

(54) COOKING MEDIUM LEVEL MONITORING SYSTEMS, METHODS, AND FRYER APPARATUS

(71) Applicants: Manouchehr SHIRALI, Eaton, OH (US); David WINTER, Eaton, OH (US); Trent ABNEY, Eaton, OH (US)

(72) Inventors: Manouchehr SHIRALI, Eaton, OH (US); David WINTER, Eaton, OH (US); Trent ABNEY, Eaton, OH (US)

(21) Appl. No.: 13/326,158

(22) Filed: Dec. 14, 2011

(51) Int. Cl. A47J 37/12; G01N 33/02

(52) U.S. Cl. 426/231; 99/331

(57) ABSTRACT

A cooking medium level monitoring system includes a cooking vessel, which holds cooking media therein; a heating mechanism that transmits heat to cooking media in the cooking vessel in a first operation state; and temperature sensors for providing data corresponding to sensed temperatures. The temperature sensors include a first temperature sensor disposed at a first level of the cooking vessel and a second temperature sensor disposed at a second level of the cooking vessel above the first level. Further, a controller receives data from the temperature sensors, calculates a temperature differential between the first temperature sensor and the second temperature sensor, and switches to a second operation state, in which the heating mechanism is deactivated, in response to the temperature differential being greater than or equal to a first threshold and less than a second threshold.

### BACKGROUND OF THE INVENTION

1. Field of the Invention

The invention relates generally to systems and methods for monitoring the level of a cooking medium in a fryer apparatus, e.g., pressure fryers or open fryers, and such fryer apparatus.

2. Description of Related Art

Known fryer apparatus are used to cook various food products, e.g., poultry, fish, potato products, and the like. Such fryer apparatus include one or more cooking chambers, e.g., a fryer pot, which are filled with cooking media, e.g., an oil, a liquid shortening, or a meltable-solid shortening. Such fryer apparatus also include a heating mechanism, e.g., an electrical heating element, such as a heating coil, or a gas heating element, such as a gas burner and gas conveying tubes, which heat the cooking medium in the cooking chamber. After the cooking medium reaches a preset cooking temperature, the food products are placed into the cooking medium, such that the food products are cooked in the cooking medium. The amount of time sufficient to cook or to complete the cooking of the food products at a given cooking temperature depends on the type of food product that is cooked. Moreover, the cooking medium may be used during several cooking cycles before the cooking medium inside the cooking vessel is filtered, replaced, or supplemented with a new or filtered supply of cooking medium.

The cooking medium in an open-well or pressure fryer is maintained at a predetermined level to standardize or to optimize cooking performance, or both. During each cooking cycle, however, the food products may absorb an amount of cooking medium during cooking. In addition, a quantity of cooking medium also may evaporate or spill out of the cooking vessel during use. Consequently, the level of cooking medium in the cooking vessel may decrease over repeated cooking cycles.

In addition, low cooking medium levels may occur in open and pressure fryers due to system or operator faults. The low cooking medium levels may result in operating inefficiencies, equipment damage, operator inconvenience, and reduced food quality. Existing high level limits do not effectively mitigate these consequences.

### SUMMARY OF THE INVENTION

Therefore, a need has arisen for systems and methods for cooking apparatus that overcome these and other shortcomings of the related art. A technical advantage of the present invention is to eliminate or reduce undesirable consequences by detecting lower cooking medium levels and preventing heating mechanism energization when low levels are detected. Another technical advantage of the present invention is to issue an alert to the equipment operator when the cooking medium level is detected to be lower or too low. The cooking medium level monitoring systems and methods may detect lower cooking medium levels by measuring a temperature difference between a regulation temperature sensor at a lower elevation in the cooking vessel, and a lower cooking medium temperature sensor at an elevation in the cooking vessel above that of the regulation temperature sensor. Two different temperature differential thresholds may distinguish operating states with and without an operator alert, so that undesirable consequences may be prevented without spurious alerts.

In an embodiment of the invention, a cooking medium level monitoring system includes a cooking vessel configured to hold cooking media therein; a heating mechanism configured to transmit heat to cooking media in the cooking vessel in a first operation state; and a plurality of temperature sensors for providing data corresponding to sensed temperature. The plurality of temperature sensors include a first temperature sensor disposed at a first level of the cooking vessel, and a second temperature sensor disposed at a second level of the cooking vessel above the first level of the cooking vessel. The cooking medium level monitoring system further includes a controller configured to: receive data from the plurality of temperature sensors, calculate a temperature differential between a first temperature measured by the first temperature sensor and a second temperature measured by the second temperature sensor based on the received data, and switch to a second operation state in response to the temperature differential calculated to be greater than or equal to a first threshold and less than a second threshold. When the controller switches to a second operation state, the heating mechanism is deactivated.

Other objects, features, and advantages of the present invention are apparent to persons of ordinary skill in the art in view of the following detailed description of the invention and the accompanying drawings.

### BRIEF DESCRIPTION OF THE DRAWINGS

For a more complete understanding of the embodiments of the present invention, needs satisfied thereby, and the objects, features, and advantages thereof, reference now is made to the following description taken in connection with the accompanying drawings.

FIG. 1 is a front view of a fryer apparatus, according to an embodiment of the invention.

FIG. 2 is a front view of a cooking vessel implementing a cooking medium monitoring system, according to an embodiment of the invention.

FIG. 3 is an overhead perspective view of a cooking vessel implementing a cooking medium monitoring system, according to an embodiment of the invention.

FIG. 4 is a diagram of the cooking medium monitoring system, according to an embodiment of the invention.

FIG. 5 is a diagram depicting one exemplary implementation of operation of the cooking medium monitoring system, according to an embodiment of the invention.

### DETAILED DESCRIPTION OF EMBODIMENTS OF THE INVENTION

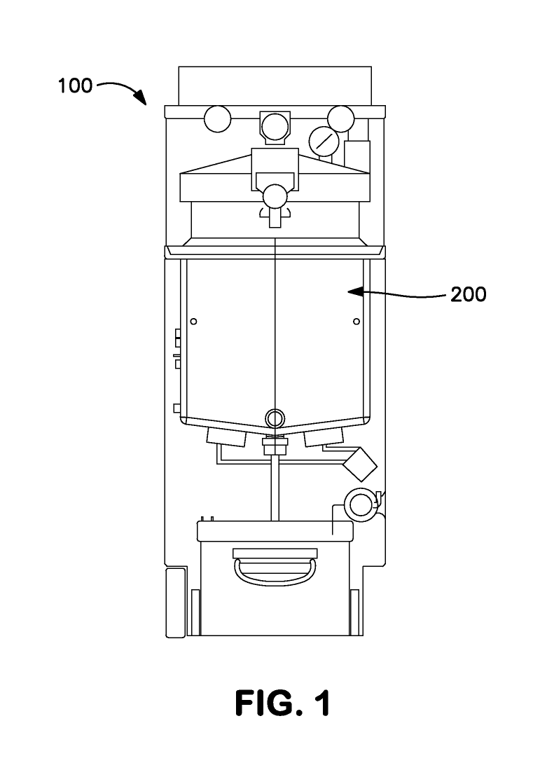

Embodiments of the present invention and their features and advantages may be understood by referring to FIGS. 1-5; like numerals being used for corresponding parts in the various drawings.

FIG. 1 depicts a fryer apparatus 100 according to an embodiment of the invention. Fryer apparatus 100 includes a cooking vessel 200. Cooking vessel 200 is configured to hold cooking media therein, and a heating mechanism may be disposed adjacent to or within cooking vessel 200.

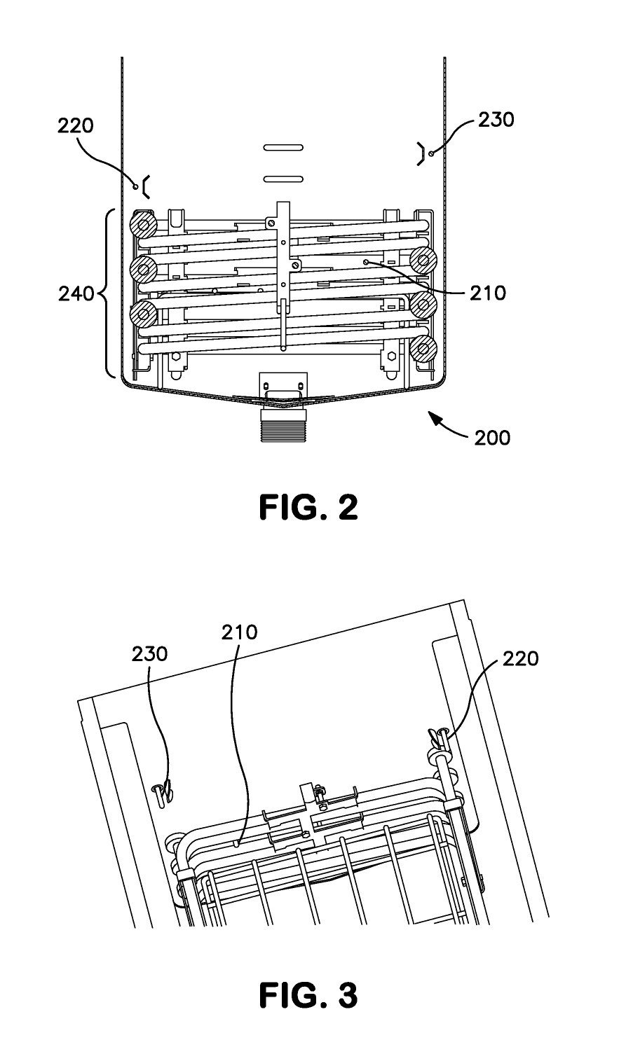

As depicted in FIGS. 2 and 3, three temperature sensors, e.g., resistance temperature detectors, resistive thermal devices (RTDs), or the like, may be mounted in cooking vessel 200 at three different elevations. A first temperature sensor 210, which may function as a regulation sensor, may be disposed at a predetermined level that is the lowest elevation of the three temperature sensors. The first temperature sensor 210 may detect a regulation temperature, T_{REG}. A second temperature sensor 220, which may function as a low cooking medium sensor, may be disposed at a middle elevation that is above the first temperature sensor 210 and below a third temperature sensor 230. The second temperature sensor 220 may detect a low temperature, T_{LOW}. The third temperature sensor 230, which may function as a top-off sensor, may be disposed at the highest elevation of the three temperature sensors. The third temperature sensor 230 may be used to implement or initiate an automatic cooking medium top-off system or give an indication that additional cooking medium should be added to the cooking vessel. In an alternative embodiment, the third temperature sensor 230 may be omitted.

A heating mechanism 240 may be disposed adjacent to or within cooking vessel 200. Heating mechanism 240 may be an electrical heating mechanism, a gas heating mechanism (e.g., a gas burner), or the like. Heating mechanism 240 may include a heating element, such as an electrical heating coil, a gas conveying tube, a heat transfer tube, or the like.

The first temperature sensor 210 may be disposed at the predetermined level in cooking vessel 200 to accomplish desired regulation of the cooking medium temperature. In an embodiment of the invention, first temperature sensor 210 may be disposed between heating elements of heating mechanism 240 to increase heat transfer from the heating mechanism 240 to the sensor through the air gap between them when neither the first temperature sensor 210 nor the heating mechanism 240 are immersed in cooking medium.

The second temperature sensor 220 may be disposed at a level above the uppermost heating element of the heating mechanism 240. The location of second temperature sensor 220 may correspond to the minimum desired cooking medium level to energize heating mechanism 240. The second temperature sensor 220 may be located a predetermined distance above the uppermost heating element of heating mechanism 240 to provide a sufficient separation for measurement. If the second temperature sensor 220 is located too close to heating mechanism 240, a low cooking medium condition then may be undetected; and the layer of cooking medium above heating mechanism 240 may be overheated when heating mechanism 240 is energized. Conversely, if the second temperature sensor 220 is located too far above the heating mechanism 240, false low cooking medium conditions then may be reported when the cooking medium level is actually sufficient.

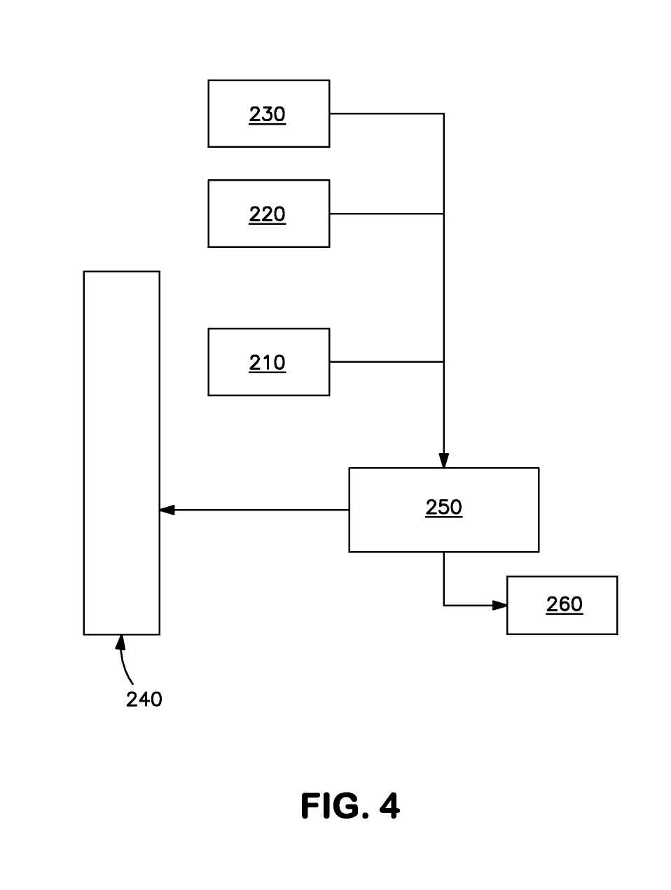

As depicted in FIG. 4, the system may include a controller 250 that may receive data from first temperature sensor 210, second temperature sensor 220, and third temperature sensor 230. Controller 250 may comprise a microprocessor and a memory. Controller 250 may be configured to calculate a temperature difference, ΔT, as follows: ΔT=T_{REG}−T_{LOW}. This temperature difference may change depending on the cooking medium level relative to the first temperature sensor 210 and the second temperature sensor 220. If the cooking medium level is sufficient to cover both the first temperature sensor 210 and the second temperature sensor 220, both sensors then may be approximately the same temperature and ΔT may be calculated to be about equal to zero. If the first temperature sensor 210 is covered by the cooking medium, but the second temperature sensor 220 is not, the temperature, T_{REG}, detected by the first temperature sensor 210 then may be higher than the temperature, T_{LOW}, detected by the second temperature sensor 220 because heat applied to the cooking medium from heating mechanism 240 may heat the cooking medium more than the air above the cooking medium. If the cooking medium level is sufficiently low that neither sensor is immersed in cooking medium, the first temperature sensor 210 may attain an elevated temperature when the heating mechanism 240 is activated because of its closer proximity to heating mechanism 240.

If one or both of the first temperature sensor 210 and the second temperature sensor 220 are uncovered, ΔT then may be substantially greater than zero. Controller 250, which may be hardware, firmware, or a combination of hardware and firmware, may be configured to measure and monitor the temperature detected by first temperature sensor 210 and the temperature detected by second temperature sensor 220, calculate ΔT, and regulate the output of heating mechanism 240 to prevent application of heat when the cooking medium is low. First temperature sensor 210 and second temperature sensor 220 may continually take a plurality of temperature readings concurrently. Alternatively, first temperature sensor 210 and second temperature sensor 220 may take a plurality of temperature readings at predetermined intervals of time.

The fryer apparatus may be in one of four states according to the cooking medium level, as determined by the calculated ΔT: (1) an initialization state (INIT) used to first determine one of the other three states when the system is powered up or resumes operation from a standby condition; (2) an operational state (NORM), in which both first temperature sensor 210 and second temperature sensor 220 are immersed in the cooking medium, may utilize heat regulation algorithms to control the heating mechanism 240 to regulate the cooking medium temperature; (3) a caution state (CAUTION), in which one or both of first temperature sensor 210 and second temperature sensor 240 is not immersed in the cooking medium, such that the heating mechanism 240 is de-energized; and (4) a warning state (WARNING), in which one or both of the first temperature sensor 210 and second temperature sensor 220 is not immersed in the cooking medium, such that the heating mechanism is de-energized and an alarm may be issued to the operator.

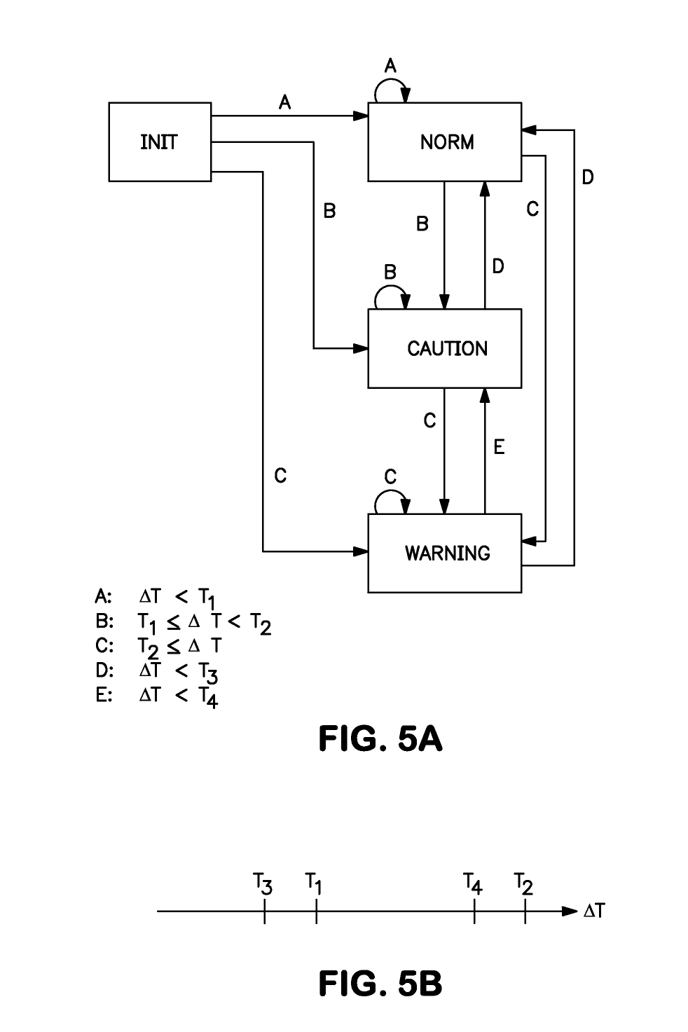

The state transition diagram of FIG. 5A depicts that the transitions between the four states depend on five ΔT conditions: (A) ΔT<T₁, (B) T₁≦ΔT<T₂, (C) T₂≦ΔT, (D) ΔT<T₃, and (E) ΔT<T₄. The ΔT thresholds: T₁, T₂, T₃, and T₄, may be determined empirically. An example of the relationships between the ΔT thresholds is depicted in FIG. 5B. In general, threshold T₁ may effect the transition from the NORM state to the WARNING state, and threshold T₂ may effect the transition from the CAUTION state to the WARNING state. T₂ may be greater than T₁, so that the WARNING state may be active at a larger ΔT than the CAUTION state. The ΔT thresholds: T₃ and T₄ may be slightly less than T₁ and T₂, respectively, to account for hysteresis. The T₃ and T₄ thresholds may prevent rapid switching between the NORM, WARNING, and CAUTION states when ΔT is near thresholds T₁ and T₂. Such rapid switching is undesirable because it may cause rapid cycling of heating mechanism 240, and the rapid switching may cause confusing activation and deactivation of the alarm issued to the operator.

The ΔT thresholds: T₁ and T₂ may be determined by testing different operating conditions of cooking medium level and temperature regulation schemes. For example, the thresholds T₁ and T₂ may be selected to allow the fryer apparatus to operate as desired regardless of the cooking medium level and heat regulation mode. The T₁ and T₂ thresholds also may depend on the geometry of the cooking vessel, cooking medium volume, sensor locations, and heating mechanism wattage. The T₁ and T₂ thresholds may be selected to achieve reliable level detection without false alarms under all operating conditions, normal and abnormal. The T₁ and T₂ thresholds also may be adjusted by criteria beyond the functional requirements described above. For example, in a particular implementation, if it is more desirable to prolong heating element life than to avoid false alarms, the threshold T₂ then may be reduced to a lower value.

For a specific fryer consisting of a cooking vessel 200, heating mechanism 240, and sensor configuration, as depicted in FIGS. 2 and 3, the ΔT thresholds may be: T₁=30.0° F. (16.7° C.), T₂=50.0° F. (27.8° C.), T₃=26.0° F. (14.4° C.), and T₄=46.0° F. (25.6° C.).

In both the CAUTION and WARNING states, the heating mechanism 240 may be de-energized. An alarm may be issued only in the WARNING state, or an alarm may be issued in both the WARNING state and the CAUTION state. In operation, because the heating mechanism 240 is de-energized in the CAUTION state, both the first temperature sensor 210 and the second temperature sensor 220 may approach equilibrium over time. If both the first temperature sensor 210 and the second temperature sensor 220 are immersed in cooking medium, then ΔT may decrease below T₃, and the controller 250 may switch from the CAUTION state to the NORM state. If the first temperature sensor 210 is immersed in cooking medium, but the second temperature sensor 220 is not immersed in cooking medium, the temperature detected by first temperature sensor 210 then may increase while the temperature detected by second temperature sensor 220 may remain relatively constant, such that ΔT increases, and the controller 250 may switch from the CAUTION state to the WARNING state. The CAUTION state may ensure that false alarms are not issued, but the heating mechanism may still be de-energized if the cooking medium level is low.

The INIT state is executed on power-up, or resuming operation from a standby condition, to select one of the three states: NORM, CAUTION or WARNING. The controller 250 does not switch back to INIT, as long as the system maintains operational power.

The format of the alarm issued to the operator may consist of an audible alarm, a visual alarm, or both. The format may vary depending on particular implementation requirements, and the format may also change during the WARNING state depending on operator interaction. For example, it may be desirable to issue both visual and audible alarm components on first entering the WARNING state, then issue only the visual component after the operator has acknowledged the alarm conditions by pressing a switch or button.

The audible and visual alarms may be implemented with variations known in the art, such as flashing an alarm LED or light, displaying the visual alarm as a symbol or text message on a display, displaying the text message in one or more local languages, and changing the frequency, volume, or pattern of the audible alarm. If the system is connected to a local- or wide-area-network, the alarm status also may be issued over that network.

While the invention has been described in connection with preferred embodiments, it will be understood by those of ordinary skill in the art that other variations and modifications of the preferred embodiments described above may be made without departing from the scope of the invention. Other embodiments will be apparent to those of ordinary skill in the art from a consideration of the specification or practice of the invention disclosed herein. The specification and the described examples are considered as exemplary only, with the true scope and spirit of the invention indicated by the following claims.

What is claimed is:

1. A cooking medium level monitoring system, comprising:
a cooking vessel configured to hold cooking media therein;
a heating mechanism configured to transmit heat to cooking media in the cooking vessel in a first operation state;
a plurality of temperature sensors providing data corresponding to sensed temperatures, comprising:
a first temperature sensor disposed at a first level of the cooking vessel, and
a second temperature sensor disposed at a second level of the cooking vessel above the first level of the cooking vessel; and
a controller configured to: receive data from said plurality of temperature sensors, calculate a temperature differential between the first temperature sensor and the second temperature sensor based on the received data, and switch to a second operation state in response to the temperature differential being calculated to be greater than or equal to a first threshold and less than a second threshold;
wherein the heating mechanism is deactivated in the second operation state.

2. The cooking medium level monitoring system of claim 1, wherein the controller is further configured to switch to a third operation state in response to the temperature differential being calculated to be greater than or equal to the second threshold; and
wherein the heating mechanism is deactivated and an alarm is activated in the third operation state.

3. The cooking medium level monitoring system of claim 1, wherein the second temperature sensor is disposed at a predetermined acceptable minimum level of cooking medium held in the cooking vessel.

4. The cooking medium level monitoring system of claim 3, wherein the predetermined acceptable minimum level of cooking medium is above the heating mechanism.

5. The cooking medium level monitoring system of claim 1, wherein the heating mechanism comprises a plurality of heating elements and the first temperature sensor is disposed between two of the plurality of heating elements.

6. The cooking medium level monitoring system of claim 2, wherein the controller is further configured to enter an initialization state in response to an applied power level changing from an off or standby level to an operational level; and
wherein, in the initialization state, the controller is configured to receive data from the plurality of temperature sensors, calculate a temperature differential between the first temperature sensor and the second temperature sensor based on the received data, and switch to one of: the first operation state in response to the temperature differential being calculated to be less than the first threshold, the second operation state in response to the temperature differential being calculated to be greater than or equal to the first threshold and less than the second threshold, and the third operation state in response to the temperature differential being calculated to be greater than or equal to the second threshold.

7. The cooking medium level monitoring system of claim 2, wherein the controller is further configured to switch from the second operation state or the third operation state to the first operation state in response to the temperature differential being calculated to be less than a third threshold, wherein the third threshold is less than the first threshold.

8. The cooking medium level monitoring system of claim 2, wherein the controller is further configured to switch from the third operation state to the second operation state in response to the temperature differential being calculated to be less than a fourth threshold, wherein the fourth threshold is less than the second threshold and greater than the first threshold.

9. The cooking medium level monitoring system of claim 1, wherein the plurality of temperature sensors further comprises a third temperature sensor disposed at a third level of the cooking vessel above the second level of the cooking vessel.

10. A fryer apparatus, comprising:
a cooking vessel configured to hold cooking media therein;
a heating mechanism configured to transmit heat to cooking media in the cooking vessel in a first operation state;
a plurality of temperature sensors providing data corresponding to sensed temperature, comprising:
a first temperature sensor disposed at a first level of the cooking vessel, and
a second temperature sensor disposed at a second level of the cooking vessel above the first level of the cooking vessel; and
a controller configured to: receive data from said plurality of temperature sensors, calculate a temperature differential between the first temperature sensor and the second temperature sensor based on the received data, and switch to a second operation state in response to the temperature differential being calculated to be greater than or equal to a first threshold and less than a second threshold;
wherein the heating mechanism is deactivated in the second operation state.

11. A method for monitoring the level of cooking media held in a cooking vessel, comprising:
receiving data from a plurality of temperature sensors, the plurality of temperature sensors comprising: a first temperature sensor disposed at a first level of the cooking vessel, and a second temperature sensor disposed at a second level of the cooking vessel above the first level of the cooking vessel;
calculating a temperature differential between the first temperature sensor and the second temperature sensor based on the received data; and
switching from a first operation state to a second operation state in response to the temperature differential being calculated to be greater than or equal to a first threshold and less than a second threshold;
wherein a heating mechanism transmits heat to the cooking media held in the cooking vessel in the first operation state, and the heating mechanism is deactivated in the second operation state.

12. The method of claim 11, further comprising:
switching to a third operation state in response to the temperature differential being calculated to be greater than or equal to the second threshold; and
wherein the heating mechanism is deactivated and an alarm is activated in the third operation state.

13. The method of claim 12, further comprising:
initializing monitoring of the level of cooking media held in the cooking vessel in response to an applied power level changing from an off or standby level to an operational level;
wherein, the initializing step comprises:
receiving data from said plurality of temperature sensors;
calculating a temperature differential between the first temperature sensor and the second temperature sensor based on the received data; and
switching to one of: the first operation state in response to the temperature differential being calculated to be less than the first threshold, the second operation state in response to the temperature differential being calculated to be greater than or equal to the first threshold and less than the second threshold, and the third operation state in response to the temperature differential being calculated to be greater than or equal to the second threshold.

14. The method of claim 12, further comprising:
switching from the second operation state or the third operation state to the first operation state in response to the temperature differential being calculated to be less than a third threshold, wherein the third threshold is less than the first threshold.

15. The method of claim 12, further comprising:
switching from the third operation state to the second operation state in response to the temperature differential being calculated to be less than a fourth threshold, wherein the fourth threshold is less than the second threshold and greater than the first threshold.
